(19) United States
(12) Patent Application Publication
(10) Pub. No.: US 2007/0264639 A1
(43) Pub. Date: Nov. 15, 2007

(54) Identification of Echinacea and its imposters using genetic variations

(75) Inventors: Nathan Zenser, Rock Hill, MO (US); Keming Song, Frontenac, MO (US); Fan Zhang, Chesterfield, MO (US)

Correspondence Address: Polsinelli Shalton Welte Suelthaus PC;100 S. Fourth Street, Suite 1100, St. Louis, MO, 63102, US

(73) Assignee: Sigma Aldrich, Co.

(21) Appl. No.: 11/431,761

(22) Filed: May 10, 2006

(51) Int. Cl. C12Q 1/68

(52) U.S. Cl. 435/6

(57) ABSTRACT

This invention provides methods, compounds, and kits for identifying Echinacea species and for distinguishing between Echinacea and imposter species based upon molecular variations. In particular, amplification primers are provided that amplify specific regions of nucleic acid sequences to generate unique amplification profiles in the different plant species. A plant may be identified by comparing its amplification profile to known amplification profiles of Echinacea and imposter species.

### FIELD OF THE INVENTION

The present invention provides compositions, methods, and kits for distinguishing between a genus of medicinal plants (Echinacea) and imposter plants and also for distinguishing among the different species of Echinacea.

### BACKGROUND OF THE INVENTION

Echinacea is a native North American wildflower that has been used medicinally for centuries. The flowers have a spiky golden-brown central cone surrounded by pink-purple petals that droop as the flower ages. The name Echinacea stems from the Greek term, echinos, meaning hedgehog, in reference to the spiky flower center. North American Plains Indians used it for many ailments, including toothaches, sore throats, snake bites, tonsillitis, coughs, and blood diseases. Today, it is used widely in North America and European as an immune tonic to relieve and shorten the duration of infections such as colds, flu, and those of the upper respiratory and lower urinary tracts. It is also applied externally as an ointment to poorly healing wounds, burns, eczema, psoriasis and herpes simplex. It has been estimated that Echinacea is the top selling herbal supplement in the United States.

Currently, three species of Echinacea (i.e., E. angustifolia, E. pallida, and E. purpurea) are used medicinally. While the pharmacologically important constituents in Echinacea have not been conclusively identified, some of the chemical compounds they posses include caffeic acid derivatives, polysaccharides, flavonoids, essential oils, polyacetylenes, and alkyamides. The presence and amounts of these biologically relevant compounds vary among the different species. For example, the roots of E. angustifolia have the highest concentration of isobutylamine, the alkylamine held responsible for the local anesthetic effects of Echinacea, whereas this compound is virtually absent in E. pallida. Similarly, E. angustifolia roots contain a number of immunostimulatory polysaccharides. In contrast, the percentage of essential oils (e.g., borneol, α-pinene, humulene, caryophyllene, etc.) is much higher in E. pallida than in the other two species. Thus, since the different species appear to have different combinations of active ingredients and may have different indications, it is important that they be correctly identified.

Due to the popularity of Echinacea, its decreasing abundance in the wild, and its long cultivation time, other plants have been fraudulently sold or passed off as Echinacea. Currently, the most common imposter is Parthenium integrifolium, which is also known as wild quinine. Although its root morphology resembles that of Echinacea, this plant lacks the biologically active ingredients of Echinacea. Thus, there is not only a need for the proper identification of the different species of Echinacea, but also a need for distinguishing between Echinacea and imposter species.

Various methods to identify or authenticate Echinacea have been developed. Methods that rely on morphological or anatomical features can only be used with intact plant material, and thus have limited utility. Chemical and chromatographic techniques that identify plants on the basis of the active ingredients are not only labor intensive, but tend to be unreliable. They are not dependable because the chemical composition of a plant can vary from grower to grower, crop to crop, and is affected by processing steps and storage conditions. Methods that exploit genetic differences, however, show the most promise because the genetic material is quite stable.

DNA fingerprinting techniques, such as random amplified polymorphic DNA (RAPD) and amplified fragment length polymorphisms (AFLP), have examined the genetic diversity and the phylogenetic relationships within the genus Echinacea. These analyses, however, were not designed to distinguish between individual species. RAPD markers have been identified that distinguish between Echinacea and P. integrifolium (Kapteyn and Simon, 2002, Trends in New Crops and New Uses, J. Janik and A Whipkey (eds.), pp. 509-513.) ASHA Press, Alexandria, VA, pp. 509-513), but since these are random primers, many bands have to be scored to unambiguously identify each species.

What is needed, therefore, is a quick, reliable, and reproducible method that will distinguish between Echinacea and imposter species and will also distinguish among the different species of Echinacea.

### BRIEF SUMMARY OF THE INVENTION

The present invention provides oligonucleotide primers, methods, and kits to distinguish between Echinacea and imposter species, as well as to identify the different species of Echinacea.

One aspect of the invention is the provision of amplification primers, each of which when used with a universal primer, amplifies a specific region of a nucleic acid sequence in one or more of the relevant plant species.

Another aspect of the invention provides methods for distinguishing between Echinacea and imposter species and for distinguishing among the different species of Echinacea. The method comprises amplifying a specific region of a nucleic acid sequence from a plant using at least one pair of amplification primers, and identifying the plant on the basis of its amplification profile in comparison to the known amplification profiles of the Echinacea and imposter species.

An additional aspect of the invention provides kits for distinguishing between Echinacea and imposter species and for distinguishing among the different species of Echinacea. A kit comprises amplification primers, instructions for use, and known amplification profiles of Echinacea and imposter species. A kit may further comprise solutions of reaction buffer, dNTP mix, divalent cation, and Taq DNA polymerase.

Other aspects and features of the invention are described in more detail herein.

### BRIEF DESCRIPTION OF THE DRAWINGS

FIG. 1 depicts an image of a gel showing the PCR products amplified by primer sets 1, 2, and 3. Presented is an image of an ethidium bromide stained 1% agarose gel. Lanes labeled 1, 2, 3, and 4 contain Echinacea angustifolia, Echinacea pallida, Echinacea purpurea, and Parthenium integrifolium, respectively. The unmarked inner lanes contain each DNA molecular size standards, with fragments ranging from 50 bp to 2,000 bp.

FIG. 2 depicts an image of a gel showing the PCR product amplified by primer set 4. Presented is a reverse image of an ethidium bromide stained 1% agarose gel. Lanes labeled 1, 2, 3, and 4 contain E. angustifolia, E. pallida, E. purpurea, and P. integrifolium, respectively. The far right lane contains a DNA molecular size standard, with fragments ranging from 50 bp to 2,000 bp.

### DETAILED DESCRIPTION OF THE PREFERRED EMBODIMENTS

Specific amplification primers have been discovered that may be used to distinguish between Echinacea and imposter species, and they may be used to distinguish among the different species of Echinacea. As detailed in the examples, primer sets consisting of one of these primers and a universal primer amplify a specific product in one or more of the species. This method permits the identification of a plant on the basis of its amplification profile in comparison to the known amplification profiles of the Echinacea and imposter species.

I. Amplification Primers

One aspect of this invention provides amplification primers that amplify a specific region of a nucleic acid sequence in one or more of the species. A primer is typically an oligonucleotide that anneals to a complementary nucleic acid sequence, and serves as a starting point for replication. During an amplification reaction, the region between the annealing sites of two primers is amplified many fold. The starting material to be amplified may be ribonucleic acid (RNA) or deoxyribonucleic acid (DNA). In a preferred embodiment, the starting material is DNA. The DNA to be amplified in a plant species may be nuclear DNA, mitochondrial DNA, or chloroplast DNA.

Primers that amplify a specific nucleic acid sequence in select species may be designed by comparing the homologous regions of the nucleic acid sequences from the different species and looking for differences at the nucleotide level. Generally, a comparison of the homologous regions from as many relevant species as possible allows for the design of more specific primers. Upon comparison of the sequences, there may be a significant number of substitutions, an insert, or a deletion in one sequence relative to the others such that a primer may be designed to amplify a product in one but not the others, or vice versa. A pair of primers may be designed to amplify a specific product in one or more of the species, or a single primer may be designed that, when used with a universal primer, will amplify a specific product in one or more of the species. Furthermore, primers may be designed such that more than one pair may be used simultaneously to amplify products of different sizes in the different species.

The region to be amplified may be within any region of a nucleic acid sequence, provided that some of its sequence is known in several species. The amplified region may be within a protein coding gene, an RNA-coding gene, a non-coding region of DNA, a transcribed spacer region, or a non-transcribed spacer region. In one embodiment, the amplified region is within a protein-coding gene. In another embodiment, the amplified region is within the transcribed region of ribosomal RNA genes. In yet another embodiment, the amplified region spans an intergenic spacer between transfer RNA genes.

The size of the amplification products can and will vary without departing from the scope of the invention. In general, the sizes of amplification products are typically such that they are amenable to PCR amplification. For products generated by traditional PCR, the amplification products generally are of sizes that may be quickly and easily characterized after the PCR reaction. For example, the products preferably are of sizes that may be resolved readily by gel electrophoresis. In one embodiment, the amplified products may range from about 50 bp to 5,000 bp in length. In another embodiment, the amplified products may range from about 100 bp to about 3,000 bp in length. In a preferred embodiment, the amplified products range from about 200 bp to about 1,000 bp in length.

The following oligonucleotide primers are provided by this invention: SEQ ID NO:1, SEQ ID NO:2, SEQ ID NO:3, and SEQ ID NO:4. The invention also encompasses a primer whose sequence differs from the sequence of any of the primers identified herein, but which may still hybridize to and amplify the same region of DNA. In one embodiment, the amplification primer has a nucleotide sequence that is at least 75% identical to the sequence of either SEQ ID NOs:1, 2, 3, or 4. In another embodiment, the amplification primer has a nucleotide sequence that is at least 80, 85, or 90% identical to the sequence of either SEQ ID NOs:1, 2, 3, or 4. In still another embodiment, the amplification primer has a nucleotide sequence that is at least 91, 92, or 93% identical to the sequence of either SEQ ID NOs:1, 2, 3, or 4. In an alternative embodiment, the amplification primer has a nucleotide sequence that is at least 94, 95, or 96% identical to the sequence of either SEQ ID NOs:1, 2, 3, or 4. In yet another embodiment, the amplification primer has a nucleotide sequence that is at least 97, 98, or 99% identical to the sequence of either SEQ ID NOs:1, 2, 3, or 4.

Conventional algorithms may be used to determine the percent of nucleotide sequence identity between different nucleic acid sequences. In particular, the percent of identity between two nucleic acid sequences is determined using the algorithm of Karlin and Altschul (Proc. Natl. Acad. Sci. USA 87:2264-2268, 1993). Such an algorithm is incorporated into the NBLAST and XBLAST programs of Altschul et al. (J. Mol. Biol. 215:403-410, 1990). BLAST nucleotide searches may be performed with the NBLAST program to obtain nucleotide sequences homologous to the primers of the invention. See http://www.ncbi.nim.nih.gov for more details.

Hybridization of an oligonucleotide probe to a nucleic acid sample is typically performed under stringent conditions. Nucleic acid duplex or hybrid stability is expressed as the melting temperature or Tₘ, which is the temperature at which a probe dissociates from a target DNA. The Tₘ for perfectly matched hybrids may be estimated using the following equation: Tₘ=81.5−16.6(log₁₀[Na⁺])+0.41(% G+C)−(600/N), where N=length of the probe in nucleotides. This equation generally predicts the Tₘ reasonably well for oligonucleotides as long as about 70 nucleotides and as short as about 14 nucleotides. The Tₘ for imperfectly matched hybrids may be estimated by subtracting about 1° C. from the calculated Tₘ for each 1% of mismatch. For example, if a sequence having about 95% identity with the primer is sought, the Tₘ is decreased by approximately 5° C. In practice, the change in Tₘ may be between 0.5-1.5° C. per 1% mismatch. When using oligonucleotides as probes, the selected hybridization conditions are typically stringent enough to guarantee specificity, but are flexible enough to allow formation of stable hybrids at an acceptable rate. Typically, hybridizations with oligonucleotides are performed at about 5-10° C. below the Tₘ of the perfect hybrid. Thus, oligonucleotide hybridizations are typically performed in 5×SSC/5×Denhardt's solution/1.0% SDS, and the washes are done in 2×SSC/0.1% SDS at the adjusted Tₘ. The parameters of salt concentration and temperature may be varied during the washes to achieve the optimal level of hybridization between the probe and the subject nucleotide sequence. Additional guidance regarding such conditions is readily available in the art, for example, by Sambrook et al., 1989, Molecular Cloning, A Laboratory Manual, Cold Spring Harbor Press, N.Y.; and Ausubel et al., (eds.), 1995, Current Protocols in Molecular Biology, (John Wiley & Sons, N.Y.) at Unit 2.10.

The invention also includes a primer that is essentially identical in sequence to any of those disclosed herein, but which further comprises one or more restriction endonuclease recognition sequences added to either end of the primer. Similarly, the invention encompasses a primer that is essentially identical in sequence to any of those disclosed herein, but which further comprises from about 1 to about 20 nucleotides added to either end of the primer.

The invention encompasses amplification primers with standard nucleobases (adenosine, guanosine, cytosine, and thymidine), as well as primers with nonstandard nucleobases. Non-limiting examples of nonstandard nucleobases include inosine, xanthosine, isoguanosine, isocytidine, diaminopyrimidine, and deoxyuridine. An amplification primer may contain standard and/or nonstandard nucleotides linked by phosphothioate linkages, as well as phosphodiester linkages. The invention also includes amplification primers with modified or derivatized nucleotides. Non-limiting examples of modifications on the ribose or base moieties of nucleotide molecules include the addition (or removal) of acetyl groups, amino groups, carboxyl groups, carboxymethyl groups, hydroxyl groups, methyl groups, phosphoryl groups, and thiol groups. In particular, included are 2′-O-methyl and LNA nucleotides. Suitable examples of derivatized nucleotides include those with covalently attached dyes, such as fluorescein- or rhodamine-based fluorescent dyes or quenching dyes, or other molecules, such as biotin or digoxygenin.

In a preferred embodiment, SEQ ID NOs:1-4 comprise standard, non-modified, non-derivatized deoxyribonucleotides linked by phosphodiester bonds that, in conjunction with a universal primer, amplify DNA products ranging in size from about 200 bp to about 1,000 bp.

II. Methods for Distinguishing Between Echinacea and Imposter Species and for Distinguishing Among the Different Species of Echinacea

Another aspect of the invention includes methods for determining whether a plant is an Echinacea species or an imposter species and for distinguishing among the different species of Echinacea. The use of this invention allows for the authentication of medicinal plants, as well as the quality control and standardization of herbal medicines.

(a) Echinacea Species

The present invention provides a method for distinguishing among the different species of Echinacea on the basis of the different amplification profiles produced by the amplification primers (as shown in Example 2). Thus, this invention provides a means to identify the different species of Echinacea. The method may be used to distinguish among the species of Echinacea used most frequently for medicinal purposes, i.e., E. angustifolia (narrow-leaved purple coneflower), E. pallida (pale purple coneflower) and E. purpurea (purple coneflower). The method may be utilized to identify other species of Echinacea, such as, E. atrorubens (Topeka purple coneflower), E. laevigata (smooth purple coneflower), E. paradoxa (yellow coneflower), E. sanguinea (sanguine purple coneflower), E. simulata (wavy-leaved or glade coneflower), and E. tennesseensis (Tennessee coneflower). Since E. laevigata and E. tennesseensis are currently on the federal list of endangered plants, this invention also may be useful for conservation and land management.

(b) Imposter Species

The invention provides a method for distinguishing between Echinacea species and imposter species on the basis of the different amplification profiles produced by the amplification primers. As demonstrated in Example 2, the method of the invention distinguishes between Echinacea species and the imposter, Parthenium integrifolium. The method of the invention may also allow discrimination between Echinacea species and other imposter species. Non-limiting examples of imposter species include Eryngium spp. (snakeroot), Helianthus spp. (sunflowers), Lespedeza capitata (bushclover), Mahonia aquifolium (Oregon grape), and Rudbeckia spp. (coneflowers).

(c) Plant Material

Plant materials of all kinds are appropriate sources of nucleic acids for practice of this invention. The plant material may be from cultivated or wild stocks. The plant material may be fresh, dried, frozen, or stored in an aqueous solution. The plant material may be derived from roots, rhizomes, stems, leaves, leaflets, flowers, seed heads, or seeds. The plant material may be whole, shredded, sliced, diced, crushed, and pulverized, or extracted juice. The plant material may be further processed and be in the form of capsules, tablets, powders, teas, aqueous extracts, alcohol extracts, glycerine extracts, creams, or gels.

Nucleic acids may be isolated from plant materials using methods well known in the art. In a preferred embodiment, DNA is extracted from roots using standard procedures. The DNA may be highly purified or not very well purified, which means that other plant-based compounds may be present, as long as they do not interfere with the amplification reaction.

(d) Amplification of Specific Regions

The present invention provides individual oligonucleotide primers that, when used with a universal primer, amplify a specific region of DNA in one or more of the relevant species. In one embodiment, SEQ ID NO:1 may be used with a primer to a conserved region of the ITS sequence, an example is the universal ITS-4 primer (SEQ ID NO:5) to amplify a specific product in Echinacea. In another embodiment, SEQ ID NO:2 may be used with a primer to a conserved region of the ITS sequence, an example is the ITS-4 primer (SEQ ID NO:5) to amplify a specific product in Echinacea. In an alternate embodiment, SEQ ID NO:3 may be used with a primer to a conserved region of the ITS sequence, an example is the ITS-4 primer (SEQ ID NO:5) to amplify a specific product in P. integrifolium. In yet another embodiment, SEQ ID NO:4 may be used with a primer to a conserved region of trnH, an example is the trnHf primer (SEQ ID NO:6) to amplify a specific product in E. pallida. The primers or primer pairs also may be used in combination for multiplex PCR. In one embodiment, multiplex PCR may be performed with SEQ ID NOs:2, 3, and 5. In another embodiment, multiplex PCR may be performed with SEQ ID NOs:1, 3, 4, 5, and 6. In yet another embodiment, multiplex PCR may be performed with SEQ ID NOs:2, 3, 4, 5, and 6.

The amplification reaction may be performed using traditional PCR or real time PCR. In one embodiment, traditional PCR is performed with the above-mentioned primer pairs using conditions well known in the art (see the examples). The PCR products may be resolved on 1-2% agarose gels or 8-15% polyacrylamide gels and visualized by staining with fluorescent DNA-binding dyes, such as ethidium bromide or SYTOX Green. In another embodiment, real time PCR is performed using the above-mentioned primers that have been derivatized with a fluorescent dye (and a quencher dye). Fluorescent dyes with different emission spectra may be attached to the different primers for multiplex PCR. The amplified products may be quantified using methods known to a skilled artisan.

(e) Identification Based Upon Distinct Amplification Profiles

Each primer pair amplifies a specific region of DNA to generate a unique product in one or more of the relevant species. Thus, distinct amplification profiles may be generated in each species by using one or more pairs of primers. This information may then be used to identify a test plant. By comparing the amplification profile of the test plant with know amplification profiles, the plant may be identified as one of the Echinacea species or an imposter species. Furthermore, it is also possible that the amplification profile of the test plant may be a blend of profiles, indicating that it is a mixture of Echinacea species or a mixture of Echinacea and imposter species.

In a preferred embodiment the following primer pairs may be used simultaneously (i.e., multiplex PCR) or sequentially: SEQ ID NO: 1 and 5; SEQ ID NO: 3 and 5; and SEQ ID NO: 4 and 6. In another preferred embodiment the following primer pairs may be used simultaneously (i.e., multiplex PCR) or sequentially: SEQ ID NO: 2 and 5; SEQ ID NO: 3 and 5; and SEQ ID NO: 4 and 6. After the products are resolved by agarose gel electrophoresis, the identity of the plant may be determined by comparing its amplification profile with known amplification profiles of the Echinacea and imposter species.

III. Kits for Distinguishing Between Echinacea and Imposter Species and for Distinguishing Among the Different Species of Echinacea

Another aspect of the invention encompasses kits for determining whether a plant is an Echinacea species or an imposter species and for distinguishing among the different species of Echinacea. In general, a kit comprises amplification primers, instructions for use, and known amplification profiles of Echinacea and imposter species. In one embodiment, the amplification primers comprise SEQ ID NOs:1-4. In another embodiment, the amplification primers comprise SEQ ID NOs:1-6. In other embodiments, the kit may further comprise solutions of reaction buffer, dNTPs, MgCl₂, and Taq DNA polymerase for the amplification reaction.

Definitions

The term “amplification primer” used herein refers to an oligonucleotide (a short strand of nucleotides) that anneals to a complementary nucleic acid sequence and serves as a starting point for an amplification reaction, whereby the region between two primer annealing sites is amplified many fold.

The term “amplification profile” used herein refers to the distinct set of products amplified in each plant species.

As used herein, the terms “imposter” or “imposter species” refer to a plant that may be marketed or passed off as Echinacea. While an imposter may lack the active ingredients found in Echinacea and be innocuous, it may also contain other (potentially harmful) active ingredients.

The term “universal primer” refers to an oligonucleotide primer whose sequence is in the public database and may be commercially available.

As various changes could be made in the above compounds, methods, and products without departing from the scope of the invention, it is intended that all matter contained in the above description and in the examples given below, shall be interpreted as illustrative and not in a limiting sense.

### EXAMPLES

The following examples illustrate various embodiments of the invention.

### Example 1

### Primer Design

There are currently three commercially relevant Echinacea species: E. angustifolia (narrow-leaved purple coneflower), E. pallida (purple coneflower), and E. purpurea (pale purple coneflower). A common imposter mistaken for Echinacea is Parthenium integrifolium (wild quinine). PCR amplification primers that would generate unique sets of DNA fragments in the different species were designed by comparing homologous regions of DNA from these four species. For, example, the internal transcribed spacer (ITS) regions of nuclear-encoded ribosomal RNA genes were compared and regions that differed between Echinacea species and P. integrifolium were identified. Three primers, SEQ ID NOs:1-3, were designed using these sequences (see Table 1). Additionally, the psbA-trnH intergenic spacer regions of the four sequences were aligned and a primer unique to E. pallida, SEQ ID NO:4, was designed (see Table 1). The primers were synthesized by Sigma-Genosys.

TABLE 1

Primer names and sequences.

| | | |
|---|---|---|
| Primer name | Sequence | SEQ ID NO |

| | | |
|---|---|---|
| EC1 | CAACCCCCGGCACAAAAT | 1 |
| EC2 | TGCATACTGTGCGTTGCTT | 2 |
| Q1 | ATGGCTGCACCATACATAT | 3 |
| psbAv | TAACGCCCTCTTGTTTTAT | 4 |
| ITS-4 | TCCTCCGCTTATTGATATGC | 5 |
| trnHf | CGCGCATGGTGGATTCACAATCC | 6 |

### Example 2

### PCR to Distinguish Echinacea Species from the Imposter Species, and to Distinguish Among the Echinacea Species

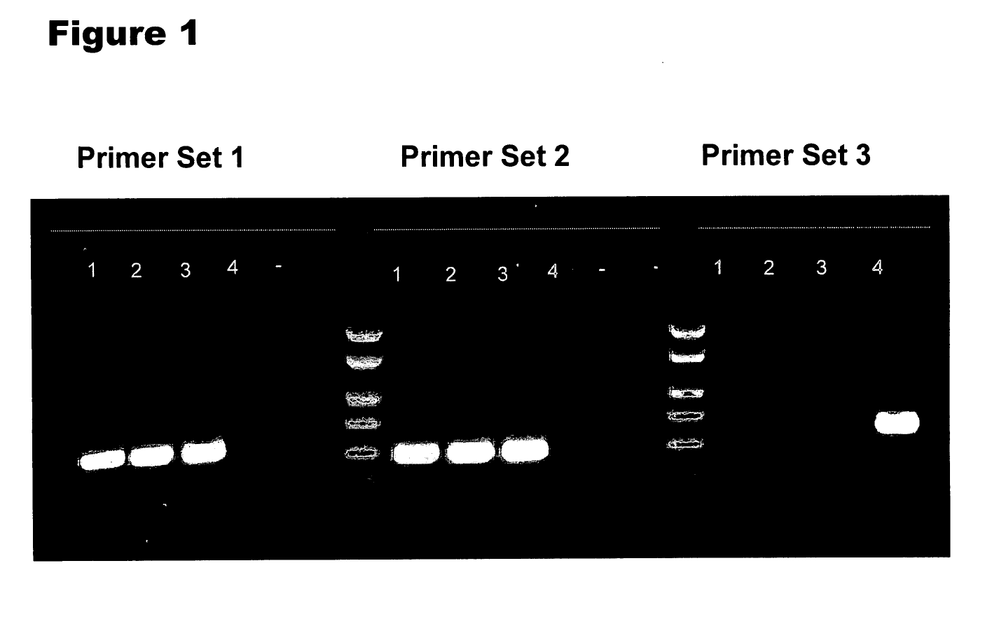

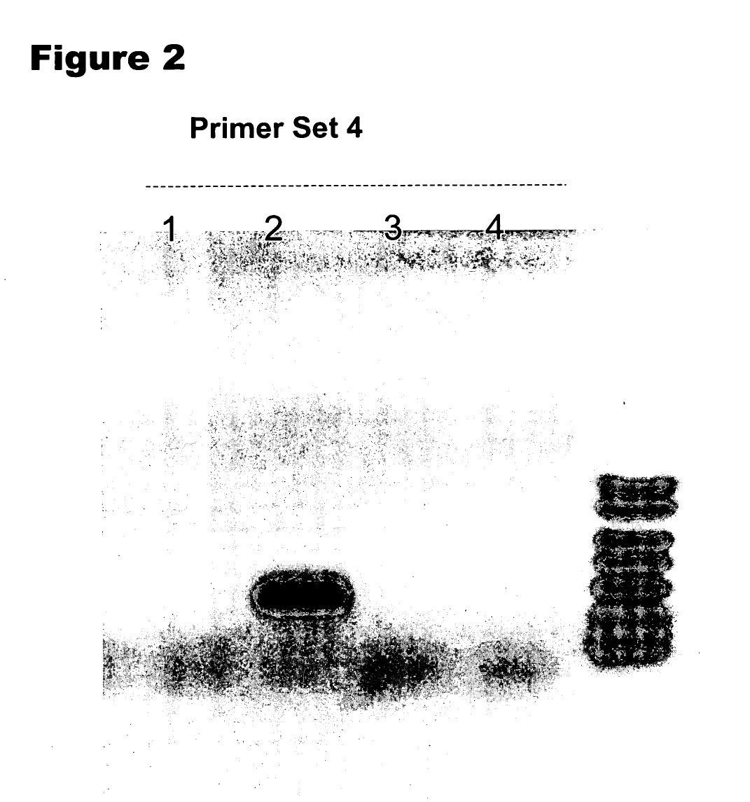

Each of the above-mentioned primers was used for PCR in conjunction with the universal ITS-4 primer (SEQ ID NO:5) or the universal primer trnHf (SEQ ID NO:6). Table 2 presents the primer pair combinations. A typical PCR reaction (20 μl) contained 10 μl of 2× JumpStart REDTaq ReadyMix (P0982, Sigma-Aldrich, St. Louis, Mo.), 1 μl of 10 μM primer set, and 25 ng of genomic DNA from the individual plant species. The cycling parameters were 94° C. for 3 min, 30 cycles of 94° C. for 15 sec, 55° C. for 30 sec, and 68° C. for 30 sec, followed by 68° C. for 7 min and 4° C. indefinitely. Typically, a 5 μl, aliquot of each reaction was resolved on a 1% TBE/agarose gel. Each primer set amplified a specific product in one or more species (see FIGS. 1 and 2). These data reveal that the PCR primers described in this invention may be used to generate specific DNA products in certain species. Furthermore, the presence or absence of these products may be used to distinguish species of Echinacea from the imposter species and to identify the different species of Echinacea.

TABLE 2
Composition of the primer sets.

| | | Plant(s) with amplified |
|---|---|---|
| Primer set | Primer pairs | product |
| 1 | SEQ ID NO: 1 | Echinacea spp. |
| | SEQ ID NO: 5 | |
| 2 | SEQ ID NO: 2 | Echinacea spp. |
| | SEQ ID NO: 5 | |
| 3 | SEQ ID NO: 3 | P. integrifolium |
| | SEQ ID NO: 5 | |
| 4 | SEQ ID NO: 4 | E. pallida |
| | SEQ ID NO: 6 | |

What is claimed is:

1. A method for determining whether a plant is an Echinacea species or an imposter species, the method comprising:
a. amplifying a specific region of a nucleic acid sequence from the plant using at least one pair of amplification primers, whereby the primers generate a specific amplification profile in each species; and
b. identifying the plant by comparing its amplification profile against known amplification profiles of Echinacea and imposter species.

2. The method of claim 1, wherein the pair of amplification primers comprises a first primer selected from the group consisting of SEQ ID NOs:1, 2, 3, and 4, and a second primer that is a specific universal primer.

3. The method of claim 2, wherein the first primer is at least seventy-five percent identical in nucleotide sequence to SEQ ID Nos:1, 2, 3, or 4 and the second primer is at least seventy-five percent identical in nucleotide sequence to the specific universal primer.

4. The method of claim 1, wherein the nucleotides comprising the primers are selected from the group consisting of standard, nonstandard, modified, and derivatized nucleotides.

5. The method of claim 1, wherein the Echinacea species is selected from the group consisting of E. angustifolia, E. pallida, E. purpurea, E. afrorubens, E. laevigata, E. paradoxa, E. sanguinea, E. simulata, and E. tennesseensis.

6. The method of claim 1, wherein the imposter species is selected from the group consisting of Parthenium integrifolium, Eryngium spp., Helianthus spp., Lespedeza capitata, Mahonia aquifolium, and Rudbeckia spp.

7. The method of claim 1, wherein the Echinacea species is selected from the group consisting of E. angustifolia, E. pallida, and E. purpurea, and the imposter species is Parthenium integrifolium.

8. The method of claim 1, wherein the plant is a material selected from the group consisting of roots, rhizomes, stems, leaves, leaflets, flowers, seed heads, and seeds.

9. The method of claim 8, wherein the plant material is selected from the group consisting of whole, shredded, sliced, diced, crushed, pulverized material, and extracted juice.

10. The method of claim 1, wherein the plant is a processed plant material selected from the group consisting of capsules, tablets, powders, teas, aqueous extracts, alcohol extracts, glycerine extracts, creams, and gels.

11. The method of claim 1, wherein the nucleic acid sequence is deoxyribonucleic acid.

12. The method of claim 1, wherein the nucleic acid sequence is amplified using PCR.

13. The method of claim 1, wherein the nucleic acid sequence is amplified using real time PCR.

14. The method of claim 1, wherein the method further comprises distinguishing among species of Echinacea.

15. A method for distinguishing among species of Echinacea, the method comprising:
a. amplifying a specific region of DNA from a plant using a first amplification primer selected from the group consisting of SEQ ID NOs:1, 2, and 4 and a second amplification primer that is a specific universal primer, whereby the primers generate a specific amplification profile in each species; and
b. identifying the plant by comparing its amplification profile against known amplification profiles of Echinacea species.

16. The method of claim 15, wherein the first primer is at least seventy-five percent identical in nucleotide sequence to SEQ ID Nos:1, 2, 3, or 4 and the second primer is at least seventy-five percent identical in nucleotide sequence to the specific universal primer.

17. A single stranded oligonucleotide consisting essentially of a nucleotide sequence selected from the group consisting of SEQ ID NOs:1, 2, 3, and 4.

18. The oligonucleotide of claim 16, wherein the oligonucleotide is at least seventy-five percent identical in nucleotide sequence to SEQ ID NOs:1, 2, 3, or 4.

19. An isolated nucleic acid selected from the group consisting of SEQ ID NOs:1, 2, 3, and 4.

20. The nucleic acid of claim 19, wherein the nucleic acid is at least seventy-five percent identical in nucleotide sequence to SEQ ID NOs:1, 2, 3, or 4.

21. A kit for determining whether a plant is an Echinacea species or an imposter species, the kit comprising:
a. amplification primers that, in conjunction with specific universal primers, produce a specific amplification profile in each species;
b. instructions for amplifying a region of deoxyribonucleic from the plant; and
c. amplification profiles from Echinacea and imposter species for identifying the plant by comparing its amplification profile with the standard profiles.

22. The kit of claim 21, wherein the amplification primers are selected from the group consisting of SEQ ID NOs:1, 2, 3, and 4.

23. The kit of claim 22, wherein the primers are at least seventy-five percent identical in nucleotide sequence to SEQ ID NOs:1, 2, 3, or 4.

24. The kit of claim 21, wherein the nucleotides comprising the primers are selected from the group consisting of standard, nonstandard, modified, and derivatized nucleotides.

25. The kit of claim 21, wherein the Echinacea species is selected from the group consisting of E. angustifolia, E. pallida, E. purpurea, E. atrorubens, E. laevigata, E. paradoxa, E. sanguinea, E. simulata, and E. tennesseensis.

26. The kit of claim 21, wherein the imposter species is selected from the group consisting of Parthenium integrifolium, Eryngium spp., Helianthus spp., Lespedeza capitata, Mahonia aquifolium, and Rudbeckia spp.

27. The kit of claim 21, wherein the Echinacea species is selected from the group consisting of E. angustifolia, E. pallida, and E. purpurea, and the imposter species is Parthenium integrifolium.

28. The kit of claim 21, wherein the plant is a material selected from the group consisting of roots, rhizomes, stems, leaves, leaflets, flowers, seed heads, and seeds.

29. The kit of claim 28, wherein the plant material is selected from the group consisting of whole, shredded, sliced, diced, crushed, pulverized material, and extracted juice.

30. The kit of claim 21, wherein the plant is a processed plant material selected from the group consisting of capsules, tablets, powders, teas, aqueous extracts, alcohol extracts, glycerine extracts, creams, and gels.

31. The kit of claim 21, wherein the deoxyribonucleic acid is amplified using PCR.

32. The kit of claim 21, wherein the deoxyribonucleic acid is amplified using real time PCR.

33. The kit of claim 21, wherein the kit further comprises a reaction buffer solution, a dNTP mixture, a divalent cation solution, and Taq DNA polymerase.

34. The kit of claim 21, wherein the kit further comprises distinguishing among species of Echinacea.


SEQUENCE LISTING (6 sequences)

SEQ ID NO: 1 - DNA, 18 bp, Artificial
  PRIMER
caacccccggcacaaaat

SEQ ID NO: 2 - DNA, 19 bp, Artificial
  PRIMER
tgcatactgtgcgttgctt

SEQ ID NO: 3 - DNA, 19 bp, Artificial
  PRIMER
atggctgcaccatacatat

SEQ ID NO: 4 - DNA, 19 bp, Artificial
  PRIMER
taacgccctcttgttttat

SEQ ID NO: 5 - DNA, 20 bp, Artificial
  ITS-4 PRIMER
tcctccgcttattgatatgc

SEQ ID NO: 6 - DNA, 23 bp, Artificial
  trnHf PRIMER
cgcgcatggtggattcacaatcc